(19) United States
(12) Patent Application Publication
(10) Pub. No.: US 2025/0114119 A1
(43) Pub. Date: Apr. 10, 2025

(54) TOOL POSTURE CONTROL DEVICE AND TOOL POSTURE CONTROL METHOD THEREOF

(71) Applicant: INDUSTRIAL TECHNOLOGY RESEARCH INSTITUTE, Hsinchu (TW)

(72) Inventors: Hao-Yan WU, Tainan City (TW); An-Peng WANG, Kaohsiung City (TW); Chien-Yu WU, Kouhu Township (TW); Su-Jhen LIN, Kaohsiung City (TW)

(73) Assignee: INDUSTRIAL TECHNOLOGY RESEARCH INSTITUTE, Hsinchu (TW)

(21) Appl. No.: 18/389,013

(22) Filed: Nov. 13, 2023

(30) Foreign Application Priority Data
Oct. 5, 2023 (TW) 112138250

(51) Int. Cl. A61B 17/34; A61B 17/00; A61B 90/00

(52) U.S. Cl. CPC A61B 17/3403; A61B 17/3476; A61B 2017/00544; A61B 2017/00557; A61B 2017/3409; A61B 2090/064

(57) ABSTRACT

A tool posture control device includes a plurality of airbags, a gas supply module and a controller. A tool is allowed to be disposed among the airbags. A gas supply module connects the airbags. The controller is electrically connected to the gas supply module and configured to control the gas supply module to provide a plurality of gas to the airbags respectively according to a target bending angle value of the tool.

This application claims the benefit of Taiwan application Serial No. 112138250, filed Oct. 5, 2023, the disclosure of which is incorporated by reference herein in its entirety.

### TECHNICAL FIELD

The disclosure relates in general to a tool posture control device and a tool posture control method thereof.

### BACKGROUND

A tool posture control device may control a tool (for example, a needle or a cautery tool) to enter the interior of an object to detect a tissue inside the object. If an abnormal tissue is found, the abnormal tissue may even be processed (for example, removed, etc.). However, depending on the location of the abnormal tissue, the needle may not be able to accurately target the abnormal tissue.

### SUMMARY

According to one embodiment, a tool posture control device is provided. The tool posture control device includes a plurality of airbags, a gas supply module and a controller. A tool is allowed to be disposed among the airbags. The gas supply module is connected to the airbags. The controller electrically is connected to the gas supply module and configured to control the gas supply module to supply a plurality of gases to the airbags respectively according to a target bending angle value of the tool.

According to another embodiment, a tool posture control method is provided. The tool posture control method includes the following steps: providing a tool posture control device, wherein the tool posture control device comprises a plurality of airbags, a gas supply module and a controller, wherein a tool is allowed to be disposed among the airbags, the gas supply module is connected to the airbags, and the controller is electrically connected to the gas supply module; and controlling the gas supply module to supply a plurality of gases to the airbags respectively according to a target bending angle value of the tool by a controller.

The above and other aspects of the disclosure will become better understood with regard to the following detailed description of the preferred but non-limiting embodiment(s). The following description is made with reference to the accompanying drawings.

### BRIEF DESCRIPTION OF THE DRAWINGS

FIG. 1A illustrates a schematic diagram of a tool posture control device applied to an object according to an embodiment of the present disclosure;

FIG. 1B illustrates a schematic diagram of the tool posture control device in FIG. 1A;

FIG. 2 illustrates a schematic diagram of a functional block of the tool posture control device in FIG. 1A;

FIG. 3A illustrates a schematic diagram of the tube body and the end cap of the tool posture control device in FIG. 1 (the tool and the airbag are not illustrated);

FIG. 3B illustrates a schematic diagram of perspective view of an end of the tool posture control device in FIG. 1;

FIG. 3C and FIG. 3D illustrate a schematic diagram of the tool of the tool posture control device in FIG. 3B being bent;

FIG. 4A illustrates a schematic diagram of the dimensions of the first airbag and the second airbag of the tool posture control device in FIG. 3B;

FIG. 4B illustrates a schematic diagram of an embodiment when the volume of the first airbag in FIG. 4A increases;

FIG. 4C illustrates a schematic diagram of an embodiment when the volume of the second airbag in FIG. 4A reduces;

FIG. 5A illustrates a schematic diagram of the tube body and the end cap of a tool posture control device according to another embodiment of the present disclosure (the tool and the airbag are not illustrated);

FIG. 5B illustrates a schematic diagram of a perspective view of the end of the tool posture control device 200 in FIG. 5A;

FIG. 5C illustrates a schematic diagram of a front view of the tool posture control device in FIG. 5B;

FIG. 6A illustrates a schematic diagram of the tube body and the end cap of a tool posture control device according to another embodiment of the present disclosure (the tool and the airbag are not illustrated);

FIG. 6B illustrates a schematic diagram of a perspective view of the end of the tool posture control device in FIG. 6A;

FIG. 6C illustrates a schematic diagram of a front view of the tool posture control device in FIG. 6B; and

FIG. 7 illustrates a flow chart of a tool posture control method of the tool posture control device in FIG. 1A.

### DETAILED DESCRIPTION

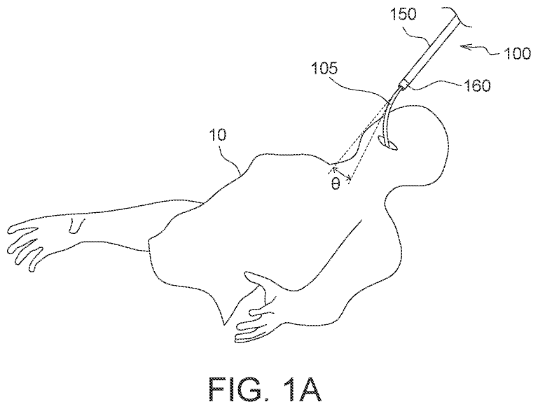

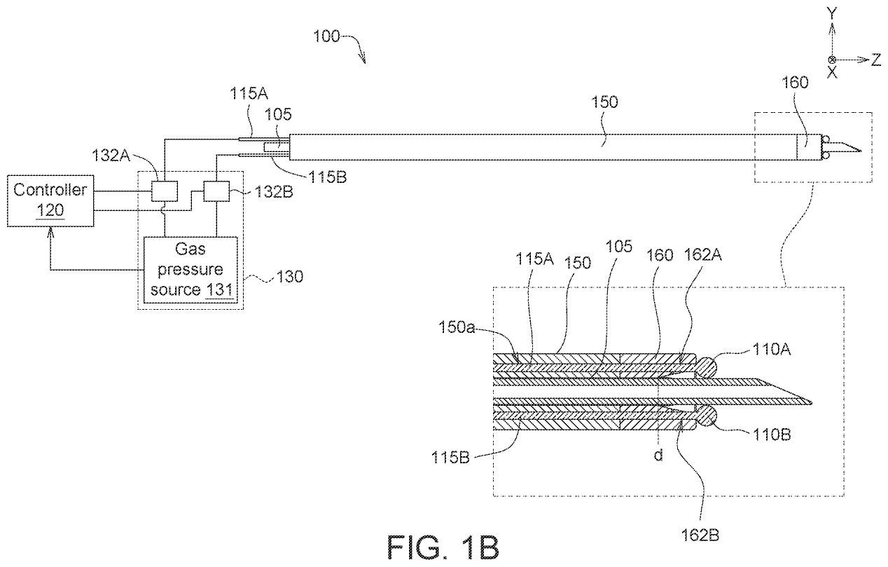

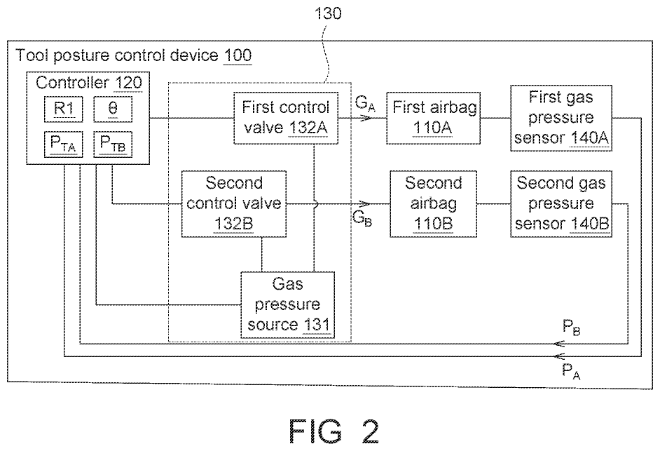

Referring to FIGS. 1A to 2, FIG. 1A illustrates a schematic diagram of a tool posture control device 100 applied to an object 10 according to an embodiment of the present disclosure, FIG. 1B illustrates a schematic diagram of the tool posture control device 100 in FIG. 1A, and FIG. 2 illustrates a schematic diagram of a functional block of the tool posture control device 100 in FIG. 1A.

As illustrated in FIG. 1A, the tool posture control device 100 may be applied in a medical field or an industrial engineering field. In the case of the medical field, the tool posture control device 100 may be applied to a robot arm. The tool posture control device 100 may control a movement of a tool 105, such as forward, backward, rotation and/or turning. The tool 105 is, for example, a needle, a cautery tool, a micro camera, a scraper (configured to scrape off foreign matter, etc.), etc. The tool 105 may enter the interior of object 10 to treat an internal structure of object 10. The object 10 may be a living body (e.g., a human or an animal, or a natural hole or channel in the living body such as a bronchi or intestine) or a non-living body (e.g., a building, a processing machine, a ground, an electronic device, a liquid tube, a gas tube, or an object other than living body). In the case of the living body, the internal structure is an abnormal tissue, for example, tumor and polyp. Although not illustrated, the tool posture control device 100 further includes a camera which may be disposed on a front end of the tool 105 or an end cap 160 to capture at least one front image, wherein the image may be transmitted to a controller 120 through wires or by using wireless communication technology. (it will be described later).

As illustrated in FIGS. 1B and 2, the tool posture control device 100 includes the tool 105, a plurality of airbags (for example, a first airbag 110A and a second airbag 110B), a plurality of gas delivery tubes (for example, a first gas delivery tube 115A and a second gas delivery tube 115B), the controller 120, a gas supply module 130, a plurality of gas pressure sensors (for example, as illustrated in FIG. 2, a first gas pressure sensor 140A and a second gas pressure sensor 140B), a pipe body 150 and the end cap 160.

As illustrated in FIGS. 1B and 2, the gas supply module 130 is connected to the first airbag 110A and the second airbag 110B. The controller 120 is electrically connected to the gas supply module 130. The tool 105 is disposed between the first airbag 110A and the second airbag 110B. The controller 120 is electrically connected to the gas supply module 130 and is configured to control the gas supply module 130 to supply a first gas G_{A} and a second gas G_{B} to the first airbag 110A and the second airbag 110B respectively according to a target bending angle value θ of the tool 105 (the target bending angle value θ is illustrated in FIG. 1A). As a result, the size (e.g., volume) of the airbag may be individually controlled through the gas for controlling (or adjusting) the bending angle (e.g., a steering and/or a turning angle) of the tool 105. In other words, the posture of the tool 105 in the embodiment of the present disclosure is controllable. Therefore, regardless of the position of the abnormal tissue relative to the tool 105, the abnormal tissue may be treated by the bending of the tool 105.

As illustrated in FIG. 1B, the first airbag 110A and the second airbag 110B are located outside the tube body 150 and the end cap 160, and leans on the end cap 160.

As illustrated in FIG. 1B, the first gas delivery tube 115A connects the first airbag 110A with a first control valve 132A, and the first gas G_{A} may be delivered to the first airbag 110A through the first control valve 132A and the first gas delivery tube 115A. The second gas delivery tube 115B connects the second airbag 110B with a second control valve 132B, and the second gas G_{B} may be delivered to the second airbag 110B through the second control valve 132B and the second gas delivery tube 115B.

As illustrated in FIGS. 1B and 2, the controller 120 is, for example, a physical circuit formed by at least one semiconductor process, such as a semiconductor chip or a semiconductor package. The controller 120 is configured to control the gas supply module 130 to supply the first gas G_{A} with a first output gas pressure value to the first airbag 110A, and to supply the second gas G_{B} with a second output gas pressure value to the second airbag 110B according to the target bending angle value θ, wherein the first output gas pressure value and the second output gas pressure value may be equal or different. By controlling the output pressure value of the gas, the volume of the airbag may be controlled, thereby controlling the bending mode of the tool 105.

As illustrated in FIGS. 1B and 2, the gas supply module 130 includes a gas pressure source 131 and a plurality of control valves (for example, the first control valve 132A and the second control valve 132B). The gas pressure source 131 is connected to the first control valve 132A with the second control valve 132B, and the gas supplied by the gas pressure source 131 may be delivered to the airbag through the control valve. The controller 120 is electrically connected to the gas pressure source 131 to control the gas pressure delivered to the control valve.

As illustrated in FIGS. 1B and 2, the first control valve 132A is connected to the first airbag 110A to be turned off or turned on for connection or disconnection of the first gas G_{A} delivered to the first airbag 110A. The second control valve 132B is connected to the second airbag 110B to be turned off or turned on for connection or disconnection of the second gas G_{B} delivered to the second airbag 110B. The controller 120 is electrically connected to the first control valve 132A and the second control valve 132B to control the control valves to be turned on or turned off. In addition, the controller 120 may control an opening level of the control valve for controlling the output gas pressure value delivered to the airbag. In addition, the controller 120 may control the gas pressure source 131 to extract the gas of the individual airbag to reduce the volume (or radius) of the airbag.

As illustrated in FIGS. 1A to 2, the controller 120 is further configured to obtain a target gas pressure value corresponding to the target bending angle value θ according to a corresponding relationship R1 between a plurality of bending angle values and a plurality of target gas pressure values. For example, in order to realize that the tool 105 presenting the target bending angle value θ, the controller 120 may obtain the gas pressure value of the first airbag 110A corresponding to the target bending angle value θ as a first target gas pressure value P_{TA} and obtain the gas pressure value of the second airbag 110B corresponding to the target bending angle value θ as a second target gas pressure value P_{TB} according to the aforementioned corresponding relationship. Therefore, the controller 120 may control the gas pressure source 131, the first control valve 132A and/or the second control valve 132B, so that the gas pressure value of the first airbag 110A is the first target gas pressure value P_{TA} and the gas pressure value of the second airbag 110B is the second target gas pressure value P_{TB}. The aforementioned corresponding relationship R1 is, for example, a table, a mathematical equation, etc. This corresponding relationship R1 may be obtained in advance through experiments or software simulation, and then stored in the controller 120, or in a memory or storage electrically connected to the controller 12.

As illustrated in FIGS. 1B and 2, the gas pressure sensors are connected to the airbags. For example, the first gas pressure sensor 140A is connected to the first airbag 110A, and the second gas pressure sensor 140B is connected to the second airbag 110B. Each gas pressure sensor is configured to sense a measured gas pressure value of the corresponding airbag, and transmit the measured gas pressure value to the controller 120. For example, the first gas pressure sensor 140A may sense a first measured gas pressure value PA of the first airbag 110A and transmit the first measured gas pressure value PA to the controller 120, while the second gas pressure sensor 140B may sense a second measured gas pressure value P_{B} of the second airbag 110B and transmit the second measured gas pressure value P to the controller 120.

In the present embodiment, the first gas pressure sensor 140A may be disposed within the first airbag 110A to sense the gas pressure value of the first airbag 110A, and the second gas pressure sensor 140B may be disposed within the second airbag 110B to sense the gas pressure value of the second airbag 110B. In another embodiment, the first gas pressure sensor 140A may be disposed within the first gas delivery tube 115A to sense the gas pressure value of the tube connected to the first airbag 110A, and the second gas pressure sensor 140B may be disposed within the second gas delivery tube 115B to sense the gas pressure value of the tube connected to the second airbag 110B. In other embodiments, the first gas pressure sensor 140A may be disposed on the first control valve 132A to sense the gas pressure value delivered to the first airbag 110A, and the second gas pressure sensor 140B may be disposed on the second control valve 132B to sense the gas pressure value delivered to the second airbag 110B. However, the arrangement position of the gas pressure sensor is not limited by the embodiments herein, as long as the gas pressure value measured by the gas pressure sensor may reflect the actual gas pressure of the airbag.

The controller 120 is further configured to control the gas supply module 130 to adjust the output value of the gas according to measured gas pressure values. Specifically, the controller 120 is further configured to: determine whether a difference between each measured gas pressure value and the corresponding target gas pressure value is outside an error range; and control the gas supply module to adjust the output value of the gas according to the measured gas pressure value whose difference is outside the error range, so that the difference falls within the error range.

For example, the controller 120 is further configured to: determine whether a first difference between the first measured gas pressure value PA of the first airbag 110A and the first target gas pressure value P_{TA} is outside the error range; and control the gas supply module 130 to adjust a first output value of the gas according to the first measured gas pressure value PA whose difference is outside the error range, so that the first difference falls within the error range. For example, when the first measured gas pressure value PA is greater than the first target gas pressure value P_{TA}, the first output value of the first gas G_{A} is reduced until the first difference falls within the error range. When the first measured gas pressure value PA is less than the first target gas pressure value P_{TA}, the first output value of the first gas G_{A} is increased until the first difference falls within the error range. Similarly, the controller 120 is further configured to: determine whether a second difference between the second measured gas pressure value P_{B} of the second airbag 110B and the second target gas pressure value P_{TB} is outside the error range; and control the gas supply module 130 to adjust a second output value of the gas according to the second measured gas pressure value P_{B} whose difference is outside the error range, so that the second difference falls within the error range. For example, when the second measured gas pressure value P_{B} is greater than the second target gas pressure value P_{TB}, the second output value of the second gas G_{B} is reduced until the second difference falls within the error range. When the second measured gas pressure value P_{B} is less than the second target gas pressure value Pre, the second output value of the second gas G_{B} is increased until the second difference falls within the error range.

In addition, the controller 120 is further configured to determine a force-applied direction of the tool 105 according to the measured gas pressure values. For example, when the tool 105 is subjected to an external force, the corresponding is squeezed. Therefore, through changes in the gas pressure value of the airbag, the force-applied direction of the tool 105 may be known.

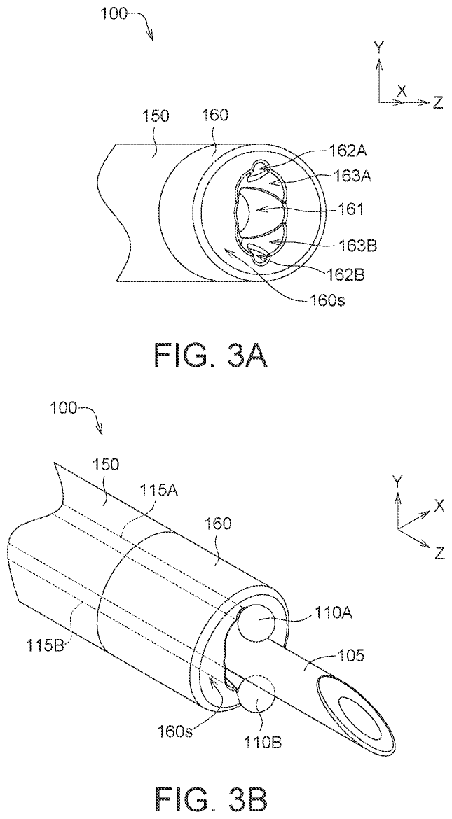

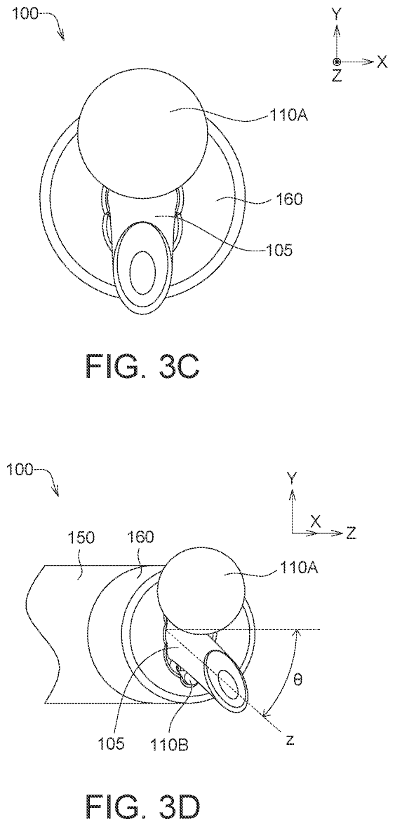

Referring to FIGS. 3A to 3D, FIG. 3A illustrates a schematic diagram of the tube body 150 and the end cap 160 of the tool posture control device 100 in FIG. 1 (the tool 105 and the airbag are not illustrated), FIG. 3B illustrates a schematic diagram of perspective view of an end of the tool posture control device 100 in FIG. 1, and FIGS. 3C and 3D illustrates a schematic diagram of the tool 105 of the tool posture control device 100 in FIG. 3B being bent.

As illustrated in FIG. 3A, the tube body 150 has a hollow portion 150a (the hollow portion 150a is illustrated in FIG. 1B), which may accommodate the aforementioned tool 105, the first gas delivery tube 115A and the second gas delivery tube 115B. The end cap 160 is disposed on the end of the tube body 150 and has an end surface 160s, a tool channel 161 and a plurality of gas delivery channels (for example, a first gas delivery channel 162A and a second gas delivery channel 162B), and the tool channel 161 and the gas delivery channels extend inward from the end face 160s.

As illustrated in FIGS. 1B and 3A to 3B, the tool 105 enters and exits the end cap 160 through the tool channel 161. Each gas delivery tube is connected to the corresponding airbag through the corresponding gas delivery channel. For example, the first gas delivery tube 115A is connected to the first airbag 110A through the first gas delivery channel 162A, and the second gas delivery tube 115B is connected to the second airbag 110B through the second gas delivery channel 162B.

As illustrated in FIG. 3A, the end cap 160 further has a plurality of position-limited grooves (for example, a first position-limited groove 163A and a second position-limited groove 163B). Each position-limited groove communicates with the tool channel 161 and extends along a movement direction to provide a receiving space for the tool 105 along a movement direction, thereby providing a degree of freedom (DoF) of movement for the tool 105 along the movement direction. For example, the first position-limited groove 163A extends from the tool channel 161 toward the +Y-axis to allow the tool 105 to bend along the +Y-axis (equivalent to the tool 105 bending around the −X-axis), while the second position-limited groove 163B extends from the tool channel 161 toward the −Y-axis to allow the tool 105 to bend along the −Y-axis (equivalent to the tool 105 bending around the +X-axis), as illustrated in FIGS. 3C and 3D. The z-axis in FIG. 3D is, for example, a central axis of the tool 105, which may represent a bending direction of the tool 105. The aforementioned target bending angle value θ is, for example, the angle between the Z-axis direction and the Z-axis direction.

As illustrated in FIGS. 3C and 3D, when the volume of the first airbag 110A is larger than the volume of the second airbag 110B, the tool 105 is squeezed to bend in the −Y-axis. In another embodiment, although not Illustrated, when the volume of the second airbag 110B is larger than the volume of the first airbag 110A, the tool 105 is squeezed to bend in the +Y-axis. In summary, the tool posture control device 100 of the embodiment of the present disclosure may provide the tool 105 with the degree of freedom to bend in the +/−Y-axis. In addition, although not illustrated, the tool 105 may rotate around the Z-axis, so that the z-axis of the tool 105 may rotate around the Z-axis.

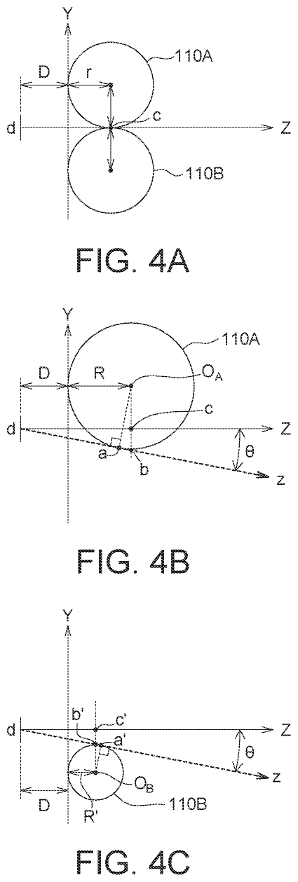

Referring to FIGS. 4A to 4C, FIG. 4A illustrates a schematic diagram of the dimensions of the first airbag 110A and the second airbag 110B of the tool posture control device 100 in FIG. 3B, FIG. 4B illustrates a schematic diagram of an embodiment when the volume of the first airbag 110A in FIG. 4A increases, and FIG. 4C illustrates a schematic diagram of an embodiment when of the volume of the second airbag 110B in FIG. 4A reduces.

As illustrated in FIG. 4A, a position d is also illustrated in FIG. 1B, which is substantially a connection between the position-limited grooves (the first position-limited groove 163A and the second position-limited groove 163B) and the tool channel 161. The tool 105 has bending freedom only in the position d. The Y-axis in FIG. 4A is the end surface 160s (the end surface 160s is illustrated in FIG. 3A) of the end cap 160. A dimension D is a distance along the Z-axis between the position d and the end face 160s, a radius r is a distance along the Z-axis between the end face 160s and a center point of the airbag, and a point c is a contact point between the two airbags.

As illustrated in FIG. 4B, when the volume of the first airbag 110A becomes larger, the radius increases to R from r. Based on the premise that Δdcb≈ΔO_{A}ab, ∠bdc=∠bO_{A}a=θ and b̅c̅=b̅O̅_̅{̅A̅}̅−O̅_̅{̅A̅}̅c̅, the following formula (1) may be derived. The symbol “≈” illustrated above is an “approximate symbol” in mathematics. The controller 120 may control the gas supply module 130 to provide the first gas G_{A} to the first airbag 110A according to formula (1), so that the first airbag 110A has the radius R to achieve the bending angle value of the tool 105 being the target bending angle value θ.

$$R = ( D sin θ + r cos θ ) / ( 1 - sin θ ) \qquad ( 1 )$$

As illustrated in FIG. 4C, when the volume of the second airbag 110B becomes smaller, the radius becomes smaller to R′ from r. Based on Δdc′b′≈ΔO_{B}a′b′, ∠b′dc′=∠b′O_{B}a′=θ and b̅′̅c̅′̅=O̅_̅{̅B̅}̅c̅′̅−b̅′̅O̅_̅{̅B̅}̅, the following formula (2) may be derived. The controller 120 may control the gas supply module 130 to provide the second gas G_{B} to the second gas G_{B} according to formula (2), so that the second airbag 110B has the radius R′ to achieve the bending angle value of the tool 105 being the target bending angle value θ.

$${R}^{′} = ( r cos θ + D sin θ ) / ( 1 - sin θ ) \qquad ( 2 )$$

The formulas (1) and (2) are applicable formulas for the two airbags. In another embodiment, an applicable formula for N airbags may be derived by using similar principles.

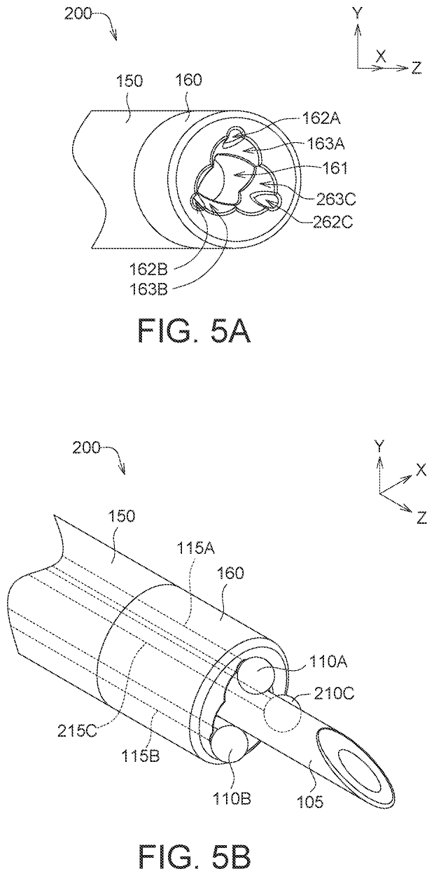

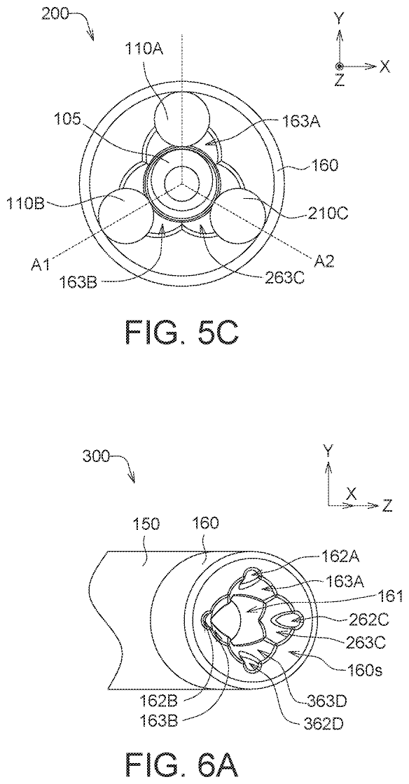

Referring to FIGS. 5A to 5C, FIG. 5A illustrates a schematic diagram of the tube body 150 and the end cap 160 of a tool posture control device 200 according to another embodiment of the present disclosure (the tool 105 and the airbag are not illustrated), FIG. 5B illustrates a schematic diagram of a perspective view of the end of the tool posture control device 200 in FIG. 5A, and FIG. 5C illustrates a schematic diagram of a front view of the tool posture control device 200 in FIG. 5B.

As illustrated in FIGS. 5A to 5B, the tool posture control device 200 includes the tool 105, a plurality of airbags (for example, the first airbag 110A, the second airbag 110B and the third airbag 210C), a plurality of gas delivery tubes (for example, the first gas delivery tube 115A, the second gas delivery tube 115B and a third gas delivery tube 215C), the controller 120, the gas supply module (not illustrated), a plurality of gas pressure sensors (not illustrated), the tube body 150 and the end cap 160.

The tool posture control device 200 includes the features (for example, structure, connection relationship, etc.) the same as or similar to that of the aforementioned tool posture control device 100, and one of the differences is that the number of gas delivery tubes of the tool posture control device 200 is different.

As illustrated in FIGS. 5A and 5B, the tube body 150 has the hollow portion 150a (illustrated in FIG. 1B) which may accommodate the tool 105, the first gas delivery tube 115A, the second gas delivery tube 115B and the third gas delivery tube 215C. The end cap 160 is disposed on the end of the tube body 150 and has the end surface 160s, the tool channel 161 and a plurality of gas delivery channels (for example, the first gas delivery channel 162A, the second gas delivery channel 162B and a third gas delivery channel 262C), and the tool channel 161 and the gas delivery channel extend inwardly from the end surface 160s.

As illustrated in FIGS. 5A and 5B, the tool 105 enters and exits the end cap 160 through the tool channel 161. Each gas delivery tube is connected to the corresponding airbag through the corresponding gas delivery channel. For example, the first gas delivery tube 115A is connected to the first airbag 110A through the first gas delivery channel 162A, the second gas delivery tube 115B is connected to the second airbag 110B through the second gas delivery channel 162B, and the third gas delivery tube 215C is connected to the third airbag 210C through the third gas delivery channel 262C.

In the present embodiment, the gas supply module includes the gas pressure source 131 and a plurality of control valves. The control valves of the gas supply module in the present embodiment have the features same as or similar to that of the control valves of the gas supply module 130 as described above, and it will not be repeated again here. The number of the control valves of the gas supply module is equal to the number of the airbags of the gas supply module in the present embodiment.

As illustrated in FIGS. 5A and 5C, the end cap 160 further has a plurality of the position-limited grooves (for example, the first position-limited groove 163A, the second position-limited groove 163B and a third position-limited groove 263C). Each position-limited groove communicates with the tool channel 161 and extends along the movement direction to provide the tool 105 with a degree of freedom of movement along the movement direction. For example, the first position-limited groove 163A extends from the tool channel 161 in the +Y-axis to allow the tool 105 to bend in the +Y-axis. The second position-limited groove 163B extends from the tool channel 161 in the first axial direction A1 to allow the tool 105 to bend in the first axial direction A1. The third position-limited groove 263C extends from the tool channel 161 in the second axial direction A2 to allow the tool 105 to move in the second axial direction A2. In an embodiment, a central angle between the first axial direction A1 and the Y-axis is, for example, 120 degrees, and the central angle between the first axial direction A1 and the second axis A2 is, for example, 120 degrees. However, such embodiment in the present disclosure is not limited to this. In an embodiment, the radius (or volume) of the first airbag 110A, the radius (or volume) of the second airbag 110B, and/or the radius (or volume) of the third airbag 210C may be controlled to control the bending direction and/or the bending angle of the tool 105.

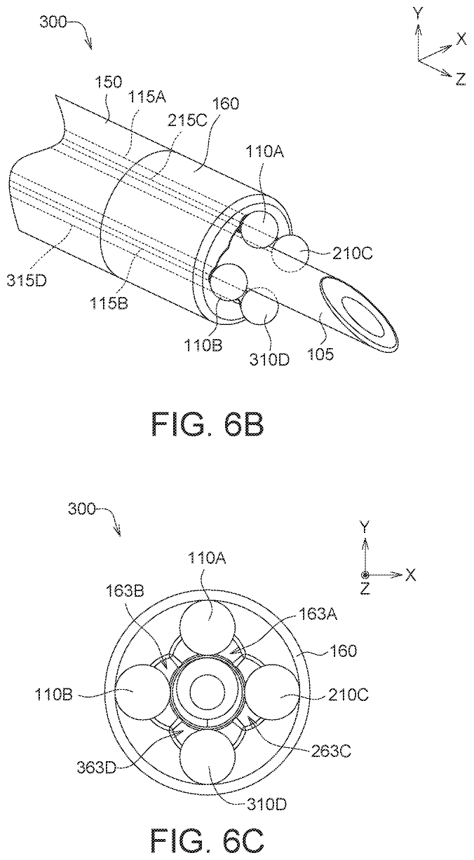

Referring to FIGS. 6A to 6C, FIG. 6A illustrates a schematic diagram of the tube body 150 and the end cap 160 of a tool posture control device 300 according to another embodiment of the present disclosure (the tool 105 and the airbag are not illustrated), FIG. 6B illustrates a schematic diagram of a perspective view of the end of the tool posture control device 300 in FIG. 6A, and FIG. 6C illustrates a schematic diagram of a front view of the tool posture control device 300 in FIG. 6B.

As illustrated in FIGS. 6A to 6B, the tool posture control device 300 includes the tool 105, a plurality of the airbags (for example, the first airbag 110A, the second airbag 110B, the third airbag 210C and a fourth airbag 310D), a plurality of the gas delivery tubes (for example, the first gas delivery tube 115A, the second gas delivery tube 115B, the third gas delivery tube 215C and a fourth gas delivery tube 315D), the controller 120, the gas supply module (not illustrated), and a plurality of the gas pressure sensors (not illustrated), the tube body 150 and the end cap 160.

The tool posture control device 300 includes the technical features (for example, structure, connection relationship, etc.) the same as or similar to that of the aforementioned tool posture control device 200. One of the differences is that the number of the gas delivery tubes of the tool posture control device 300 is different.

As illustrated in FIGS. 6A and 6B, the tube body 150 has the hollow portion 150a (illustrated in FIG. 1B) which may accommodate the tool 105, the first gas delivery tube 115A, the second gas delivery tube 115B, the third gas delivery tube 215C and the fourth gas delivery tube 315D. The end cap 160 is disposed on the end of the tube body 150 and has the end surface 160s, the tool channel 161 and a plurality of gas tube channels (for example, the first gas tube channel 162A, the second gas tube channel 162B, the third gas tube channel 262C and a fourth gas delivery channel 362D), and the tool channel 161 and the gas delivery channel extend inwardly from the end surface 160s.

As illustrated in FIGS. 6A and 6B, the tool 105 enters and exits the end cap 160 through the tool channel 161. Each gas delivery tube is connected to the corresponding airbag through the corresponding gas delivery channel. For example, the first gas delivery tube 115A is connected to the first airbag 110A through the first gas delivery channel 162A, the second gas delivery tube 115B is connected to the second airbag 110B through the second gas delivery channel 162B, the third gas delivery tube 215C is connected to the third gas delivery tube 115C through the third gas delivery channel 262C, and the fourth gas delivery tube 315D is connected to the fourth airbag 310D through the fourth gas delivery channel 362D.

In this embodiment, the gas supply module includes the gas pressure source 131 and a plurality of control valves. The control valves of the gas supply module of the present embodiment have the features same as or similar to the control valves of the gas supply module 130 described above, and it will not be repeated again here. The number of control valves of the gas supply module in the present embodiment is equal to the number of the airbags.

As illustrated in FIGS. 6A and 6C, the end cap 160 further has a plurality of the position-limited grooves (for example, the first position-limited groove 163A, the second position-limited groove 163B, the third position-limited groove 263C and a fourth position-limited groove 363D). Each position-limited groove communicates with the tool channel 161 and extends in a movement direction to provide the tool 105 with a degree of freedom of movement in the movement direction. For example, the first position-limited groove 163A extends from the tool channel 161 toward the +Y-axis to allow the tool 105 to bend along the +Y-axis. The second position-limited groove 163B extends from the tool channel 161 toward the −X-axis to allow the tool 105 to bend along the −X-axis. The third position-limited groove 263C extends from the tool channel 161 toward the +X-axis to allow the tool 105 to move along the +X-axis. The fourth position-limited groove 363D extends from the tool channel 161 toward the −Y-axis to allow the tool 105 to move along the −Y-axis.

In an embodiment, the radius (or volume) of the first airbag 110A, the radius (or volume) of the second airbag 110B, the radius (or volume) of the third airbag 210C, and/or the radius (or volume) of the fourth airbag 310D (or volume) to control the bending direction and/or the bending angle of the tool 105.

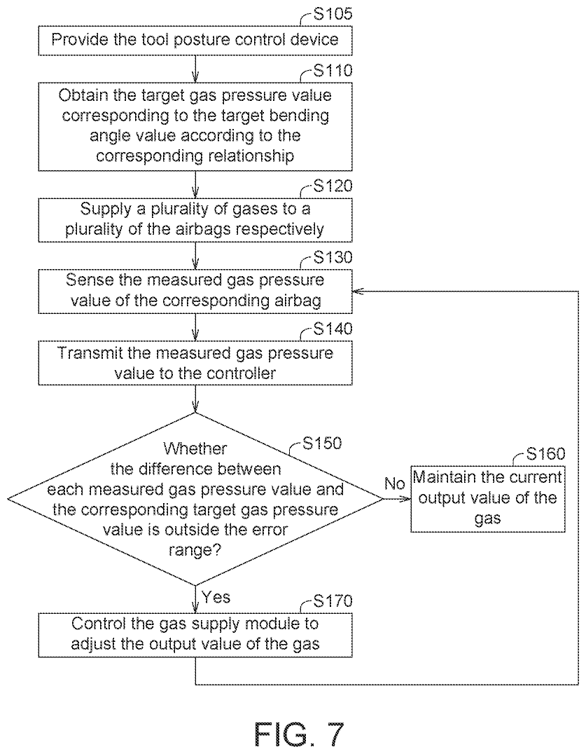

Referring to FIG. 7, FIG. 7 illustrates a flow chart of a tool posture control method of the tool posture control device 100 in FIG. 1A.

In step S105, the tool posture control device 100 as described above is provided.

In step S110, the controller 120 obtains the target gas pressure value PT corresponding to the target bending angle value θ according to the aforementioned corresponding relationship R1. In an embodiment, an operator (e.g., medical staff or engineering personnel) may transmit the target bending angle value θ to the tool posture control device 100 through a user interface (not illustrated).

In step S120, the controller 120 controls the gas supply module 130 to supply a plurality of gases to a plurality of the airbags respectively according to the target bending angle value θ of the tool. For example, as illustrated in FIG. 1B, the controller 120 may control the opening level of the first control valve 132A to supply the first gas G_{A} with the first output gas pressure value to the first airbag 110A, and control the opening level of the second control valve 132B to supply the second gas G_{B} with the second output gas pressure value to the second airbag 110B.

In step S130, each gas pressure sensor senses the measured gas pressure value of the corresponding airbag. For example, as illustrated in FIG. 1B, the first gas pressure sensor 140A senses the first measured gas pressure value PA of the first airbag 110A, and the second gas pressure sensor 140B senses the second measured gas pressure value P_{B} of the second airbag 110B.

In step S140, each gas pressure sensor transmits the measured gas pressure value to the controller 120. For example, as illustrated in FIG. 1B, the first gas pressure sensor 140A transmits the first measured gas pressure value PA to the controller 120, and the second gas pressure sensor 140B transmits the second measured gas pressure value P_{B} to the controller 120.

In step S150, the controller 120 determines whether the difference between each measured gas pressure value and the corresponding target gas pressure value is outside the error range. If so, the process proceeds to step S170; if not, the process proceeds to step S160.

For example, the controller 120 may determine whether the first difference between the first measured gas pressure value PA and the first target gas pressure value P_{TA} of the first airbag 110A is outside the error range; if so (proceeding to step S170), the controller 120 may control the gas supply module 130 to adjust the first output value of the first gas G_{A} so that the first difference falls within the error range; if not (proceeding to step S160), the current first output value of the first gas G_{A} is maintained.

Similarly, the controller 120 may determine whether the second difference between the second measured gas pressure value P_{B} and the second target gas pressure value P_{TB} of the second airbag 110B is outside the error range; if so (proceeding to step S170), the controller 120 may control the gas supply module 130 to adjusts the second output value of the second gas G_{B} so that the second difference falls within the error range; if not (proceeding to step S160), the current second output value of the second gas Ge is maintained.

Other posture control steps of the tool posture control device 100 have been described above, and it will not be repeated again here. In addition, the posture control methods of the tool posture control devices 200 and 300 are similar or same as to that of the aforementioned tool posture control device 100, and it will not be repeated again here.

In summary, embodiments of the present disclosure provide a tool posture control device. The tool posture control device includes N airbags, a tool, a gas supply module and a controller, wherein N is a positive integer equal to or greater than 1. The tool is disposed between N airbags. The gas supply module is connected to N airbags to supply N gases to the N airbags respectively. For example, the gas supply module includes N control valves, which are respectively connected to N airbags for supplying the gas to individual airbag and/or extract gas from individual airbag, thereby controlling or adjusting the radius (or volume) of the individual airbag. As a result, by controlling the radius (or volume) of the N airbags, the bending direction and/or the bending angle of the tool may be adjusted (or controlled). In an embodiment, the controller may control the gas supply module to supply the gas with the corresponding gas pressure to the airbag for achieving the aforementioned airbag radius control. In an embodiment, the central angle between adjacent two of the N airbags is approximately 360/N (degrees). In other embodiments, the tool posture control device further includes an end cap having N position-limited grooves, and the airbag may be located in the corresponding position-limited groove. In an embodiment, the central angles among the N airbags may be equal or different, and it may be achieved through the geometry (e.g., extension direction) of the N position-limited grooves of the end cap. Taking two airbags as an example, the gas supply module may supply the gas to the individual airbag and/or extract the gas from the individual airbag, thereby controlling or adjusting the radius (or volume) of the individual airbag, so that the bending angle in two axes (for example, two axes parallel to each other, but not limited to this) of the tool may be adjusted (or controlled).

It will be apparent to those skilled in the art that various modifications and variations could be made to the disclosed embodiments. It is intended that the specification and examples be considered as exemplary only, with a true scope of the disclosure being indicated by the following claims and their equivalents.

What is claimed is:

1. A tool posture control device, comprising:
a plurality of airbags, wherein a tool is allowed to be disposed among the airbags;
a gas supply module connected to the airbags; and
a controller electrically connected to the gas supply module and configured to:
control the gas supply module to supply a plurality of gases to the airbags respectively according to a target bending angle value of the tool.

2. The tool posture control device as claimed in claim 1, wherein the controller is further configured to:
control the gas supply module to supply the gas with a first output gas pressure value to one of the airbags and supply the gas with a second output gas pressure value to another of the airbags according to the target bending angle value, wherein the first output gas pressure value and the second output gas pressure value are different.

3. The tool posture control device as claimed in claim 1, wherein the controller is further configured to:
obtain a target gas pressure value corresponding to the target bending angle value according to a corresponding relationship between a plurality of bending angle values and a plurality of the target gas pressure values.

4. The tool posture control device as claimed in claim 1, further comprising:
a plurality of gas pressure sensors connected to the airbags, wherein each gas pressure sensor is configured to:
sense a measured gas pressure value corresponding to the airbag; and
transmit the measured gas pressure value to the controller;
wherein the controller is configured to:
control the gas supply module to adjust an output value of the gas according to the measured gas pressure values.

5. The tool posture control device as claimed in claim 4, wherein the controller is further configured to:
determine whether a difference between each measured gas pressure value and the corresponding target gas pressure value is outside an error range; and
control the gas supply module to adjust the output value of the gas according to the measured gas pressure value whose difference value is outside the error range.

6. The tool posture control device as claimed in claim 1, wherein the controller is further configured to:
a plurality of gas pressure sensors connected to the airbags, wherein each gas pressure sensor is configured to:
sense a measured gas pressure value of the corresponding airbag; and
transmit the measured gas pressure value to the controller;
wherein the controller is configured to:
determine a force-applied direction of the tool according to the measured gas pressure values.

7. The tool posture control device as claimed in claim 1, further comprising:
an end cap having a tool channel and a plurality of gas delivery channels; and
a plurality of gas delivery tubes each connected to the corresponding airbag;
wherein the tool enters and exits the end cap through the tool channel, and each gas tube is connected to the corresponding airbag through the corresponding gas delivery channel.

8. The tool posture control device as claimed in claim 7, wherein the end cap further has a plurality of position-limited grooves, each of the position-limited grooves is connected to the tool channel and extends along a movement direction to provide the tool with a degree of freedom of movement in the movement direction.

9. The tool posture control device as claimed in claim 7, wherein the end cap further has a plurality of position-limited grooves, and each of the position-limited grooves is configured to accommodate the corresponding airbag.

10. The tool posture control device as claimed in claim 1, further comprising:
a plurality of gas delivery tubes each connected to the corresponding airbag, and
a tube body accommodates the gas delivery tubes.

11. A tool posture control method, comprising:
providing a tool posture control device, wherein the tool posture control device comprises a plurality of airbags, a gas supply module and a controller, wherein a tool is allowed to be disposed among the airbags, the gas supply module is connected to the airbags, and the controller is electrically connected to the gas supply module; and
controlling the gas supply module to supply a plurality of gases to the airbags respectively according to a target bending angle value of the tool by a controller.

12. The tool posture control method as claimed in claim 11, further comprising:
controlling the gas supply module to supply the gas with a first output gas pressure value to one of the airbags and supply the gas with a second output gas pressure value to another of the airbags according to the target bending angle value by the controller, wherein the first output gas pressure value and the second output gas pressure value are different.

13. The tool posture control method as claimed in claim 11, further comprising:
obtaining a target gas pressure value corresponding to the target bending angle value according to a corresponding relationship between a plurality of bending angle values and a plurality of the target gas pressure values.

14. The tool posture control method according to claim 11, wherein the tool posture control device further comprises a plurality of gas pressure sensors, and the gas pressure sensors are connected to the airbags; the tool posture control method further comprises:
sensing a measured gas pressure value of the corresponding airbag by each gas pressure sensor;
transmitting the measured gas pressure value to the controller by each gas pressure sensor by the controller; and
controlling the gas supply module to adjust an output value of the gas according to the measured gas pressure values by the controller.

15. The tool posture control method as claimed in claim 14, further comprising:
determining whether a difference between each measured gas pressure value and corresponding target gas pressure value is outside an error range by the controller; and
controlling the gas supply module to adjust the output value of the gas according to the measured gas pressure value whose difference value is outside the error range by the controller.

16. The tool posture control method as claimed in claim 11, wherein the tool posture control device further comprises a plurality of gas pressure sensors, and the gas pressure sensors are connected to the airbags; the tool posture control method further comprises:
sensing a measured gas pressure value of the corresponding airbag by each gas pressure sensor;
transmitting the measured gas pressure value to the controller by each gas pressure sensor; and
determining a force-applied direction of the tool according to the measured gas pressure values by the controller.

17. The tool posture control method as claimed in claim 11, wherein the tool posture control device further comprises an end cap and a plurality of gas delivery tubes, the end cap has a tool channel and a plurality of gas delivery channels and each gas delivery tube is connected to the corresponding airbag; the tool enters and exits the end cap through the tool channel, and each gas delivery tube is connected to the corresponding airbag through the corresponding gas delivery channel.

18. The tool posture control method as claimed in claim 17, wherein the end cap further has a plurality of position-limited grooves, each of the position-limited grooves is connected to the tool channel and extends along a movement direction to provide the tool with a degree of freedom of movement in the movement direction.

19. The tool posture control method as claimed in claim 17, wherein the end cap further has a plurality of position-limited grooves, and each of the position-limited grooves is configured to accommodate the corresponding airbag.
